SEQUENCE LISTING (12 sequences)

SEQ ID NO: 1 - DNA, 17 bp, Artificial sequence
  Synthetic primer
ggaatggcggccacggg

SEQ ID NO: 2 - DNA, 21 bp, Artificial sequence
  Synthetic primer
caaggcccagctcttgcacca

SEQ ID NO: 3 - DNA, 20 bp, Artificial sequence
  Synthetic primer
gcagctactgccatccaatc

SEQ ID NO: 4 - DNA, 20 bp, Artificial sequence
  Synthetic primer
ctgcatggtgatgttggact

SEQ ID NO: 5 - DNA, 26 bp, Artificial sequence
  Synthetic primer
ctgctcgagcagcgatgcgaccctcc

SEQ ID NO: 6 - DNA, 20 bp, Artificial sequence
  Synthetic primer
acataaccagccacctcctg

SEQ ID NO: 7 - DNA, 16 bp, Artificial sequence
  Synthetic primer
ggagccgcagtgagca

SEQ ID NO: 8 - DNA, 20 bp, Artificial sequence
  Synthetic primer
gacctgcctcacttggttgt

SEQ ID NO: 9 - DNA, 19 bp, Artificial sequence
  Synthetic primer
ccatggaacaccagctcct

SEQ ID NO: 10 - DNA, 20 bp, Artificial sequence
  Synthetic primer
ggacctccttctgcacacat

SEQ ID NO: 11 - DNA, 18 bp, Artificial sequence
  Synthetic primer
cgttacttttcctctggg

SEQ ID NO: 12 - DNA, 18 bp, Artificial sequence
  Synthetic primer
ggctgcgaggagaagatg

(19) United States
(12) Patent Application Publication
(10) Pub. No.: US 2006/0037088 A1
(43) Pub. Date: Feb. 16, 2006

(54) Gene expression levels as predictors of chemoradiation response of cancer

(76) Inventor: Shulin Li, Baton Rouge, LA (US)

Correspondence Address: PATENT DEPARTMENT;TAYLOR, PORTER, BROOKS & PHILLIPS, L.L.P, P.O. BOX 2471, BATON ROUGE, LA, 70821-2471, US

(21) Appl. No.: 10/918,162

(22) Filed: Aug. 13, 2004

(51) Int. Cl. A01K 67/00; C12Q 1/68; C12M 1/34

(52) U.S. Cl. 800/10; 435/6; 435/287.2

(57) ABSTRACT

Uridine phosphorylase is a reliable molecular marker to predict the response of cancer, e.g., squamous cell carcinoma, to treatment with a chemotherapeutic agent or radiation therapy or concomitant treatment with both. Surprisingly, this molecular marker by itself is more accurate than other well-known molecular markers for the prediction of the concomitant chemoradiation response. This marker may be used as a prognostic factor on several types of cancers, including head and neck cancer, skin cancer, ovarian cancer, lung cancer, colon cancer, esophageal cancer, melanoma, and adenocarcinoma. The expression profile of eleven genes may be used to predict the response of cancer cells to chemoradiation therapy.

This invention pertains to the use of molecular markers to predict the response of cancer cells to radiation or chemotherapy, in particular, the use of the molecular marker uridine phosphorylase.

Early diagnosis is one of the keys for successful treatment of cancer. However, some cancers do not respond well to radiation, chemotherapy, or a combination of both. Many investigators have tried to identify molecular markers as prognostic factors for patients who will and will not respond to chemoradiation therapy, and as potential targets for therapeutic intervention. See B. G. Haffty et al., “Molecular markers in clinical radiation oncology,” Oncogene, vol. 22, pp. 5915-25 (2003). In general, cancer cells respond similarly to either radiation or chemotherapy. For instance, if the cells are radiation sensitive, they are also usually sensitive to chemotherapy; however, if the cells are radiation resistant, they are also usually resistant to chemotherapy. See I. Fichtner et al., “Chemo- and radiation sensitivity of xenografted acute lymphoblastic leukemias—correlation to the expression of multidrug resistance proteins,” Anticancer Res., vol. 23, pp. 2657-64 (2003); K. Kishi et al., “Prediction of the response to chemoradiation and prognosis in oesophageal squamous cancer,” Br. J. Surg., vol. 89, pp. 597-603 (2002); and C. Trejo-Becerril et al., “Correlation of tumor growth index with early treatment response in cervical carcinoma,” J. Exp. Clin. Cancer Res., vol. 21, pp. 57-63 (2002).

Early diagnosis is particularly important for successful treatment of squamous cell carcinoma (“SCC”), e.g., in the upper aerodigestive tract, whether by surgery, radiation, or chemoradiation. See P. Lavertu et al., “Aggressive concurrent chemoradiotherapy for squamous cell head and neck cancer,” Arch. Otolaryngol. Head Neck Surg., vol. 125, pp. 142-148 (1999). Patients with an advanced stage of SCC head and neck cancer have a high rate of death, with reported 5-year survival rates of 38-60% despite the development of different, aggressive, and multimodal treatments. See U.S. patent application Ser. No. 2003/0,175,717.

In an attempt to reduce mortality and preserve the function of organs in the head and neck, concomitant chemoradiation therapy has been used as an alternative treatment to surgery. One example of a combined chemoradiation treatment is to administer both fluorouracil and cisplatin with daily radiation doses (Lavertu et al., 1999). Although this combination treatment has had a relatively high success rate, some patients with SCC tumors have failed to respond. For these chemoradiation-resistant patients, surgery is the best, if not the only, effective treatment. However, the time spent in chemoradiation delays surgery, making the eventual surgery more difficult because the SCC tumor has increased in volume. The ability to predict a patient's response to chemoradiation treatment would thus be valuable: to save time for both the patient and physician, to minimize patient suffering through a non-effective treatment, and to optimize treatment strategy for each patient.

Several molecular markers have been explored to predict the radiation response of cancer cells. See U.S. patent application Ser. No. 2003/0,175,717; B. G. Haffty et al., “Molecular markers in clinical radiation oncology,” Oncogene, vol. 22, pp. 5915-25 (2003); Y. Yu et al., “Significance of c-Myc and BCL-2 protein expression in nasopharyngeal carcinoma,” Arch. Otolaryngol. Head Neck Surg., vol. 129, pp. 1322-1326 (2003); A. Gupta et al., “Local recurrence in head and neck cancer: relationship to radiation resistance and signal transduction,” Clinical Cancer Research, vol. 8, pp. 885-892 (2002); and E. Hanna et al., “A novel alternative approach for prediction of radiation response of squamous cell carcinoma of head and neck,” Cancer Research, vol. 61, pp. 2376-2380 (2001). The identified genes generally have been oncogenes, cell proliferation regulators, or cell survival genes, e.g., EGFR, Her2/neu, BCL-2, insulin-like growth factor, cyclin D, VEGF, p21, and p53.

Currently, epidermal growth factor receptor (EGFR) is reported to be the most valuable molecular marker for SCC of the head and neck. An elevated EGFR expression predicts a poor response to both radiation and chemoradiation therapy. Moreover, antibodies to EGFR have been employed concurrently with radiation to improve the response to radiation therapy. (Gupta et al., 2002; and Haffty et al., 2003).

The predictive ability of many molecule markers depends on the tumor type, the treatment history of the tumor, and other variables. For example, Her2/neu is a prognostic marker for breast cancers that are at a high risk for recurrence, and also a predictor for response to certain chemotherapy. See C. Lohrisch et al., “HER2/neu as a predictive factor in breast cancer,” Clin. Breast Cancer, vol. 2, pp. 129-135 (2001); and J. A. Carr et al., “The association of HER-2/neu amplification with breast cancer recurrence,” Arch. Surg., vol. 135, pp. 1469-1474 (2000).

Increased BCL-2 and BAX expression in radiation-treated cells from squamous cell carcinoma of the larynx predict a beneficial response to radiation therapy, while the level of expression of these genes in cells prior to radiation is not predictive of response to radiation. See L. T. Condon et al., “Overexpression of BCL-2 in squamous cell carcinoma of the larynx: A marker of radioresistance,” Int. J. Cancer, vol. 100, pp. 472-475 (2002).

Some markers have been reported to be strong predictors for recurrence of certain tumors. See A. Ringberg et al., “Cell biological factors in ductal carcinoma in situ (DCIS) of the breast-relationship to ipsilateral local recurrence and histopathological characteristics,” Eur. J. Cancer, vol. 37, pp. 1514-1522 (2001); and L. J. Pierce et al., “Is c-erb B-2 a predictor for recurrent disease in early stage breast cancer,” Int. J. Radiat. Oncol. Biol. Phys., vol. 28, pp. 395-403 (1994).

Uridine is a pyrimidine nucleoside essential for the synthesis of both RNA and biological membranes. The concentration of uridine is tightly regulated by cellular transport mechanisms and by the activity of uridine phosphorylase (UPase), an enzyme responsible for the reversible phophorolysis of uridine to uracil. See G. Pizzorno et al., “Homeostatic control of uridine and the role of uridine phosphorylase, a biological and clinical update,” Biochim. Biophys. Acta, vol. 1587, pp. 133-144 (2002).

UPase levels are elevated in many solid tumor cells, as compared to normal cells. See D. Cao et al., “Uridine Phosphorylase (−/−) murine embryonic stem cells clarify the key role of this enzyme in the regulation of the pyrimidine salvage pathway and in the activation of fluoropyrimidines,” Cancer Research, vol. 62, pp. 2313-2317 (2002); and A. Kanzaki et al., “Expression of uridine and thymidine phosphorylase genes in human breast carcinoma,” Int. J. Cancer, vol. 97, pp. 631-635 (2002).

Researchers have tried altering the expression of UPase in cancer cells to increase the response to chemotherapy with fluoropyrimidines, but the results have been inconsistent. Some studies report that experimentally-induced overexpression of UPase does not increase tumor sensitivity to fluoropyrimidines. See, e.g., P. Cuq et al., “Fluoropyrimidine sensitivity of human MCF-7 breast cancer cells stably transfected with human uridine phophorylase,” Br. J. Cancer, vol. 84, pp. 1677-1680 (2001); O. M. Ashour et al., “Enhancement of 5-fluoro-2′-deoxyuridine antitumor efficacy by the uridine phosphorylase inhibitor 5-(benzyloxybenzyl) barbituric acid acyclonucleoside,” Cancer Res., vol. 55, pp. 1092-1098 (1995); L. K. Yee et al., “Benzylacyclouridine enhances 5-fluorouracil cytotoxicity against human prostate cancer cell lines,”: Pharmacology, vol. 56, pp. 80-91 (1998); and J. W. Darnowski et al., “Enhancement of fluorouracil therapy by the manipulation of tissue uridine pools,” Pharmacol. Ther., vol. 41, pp. 381-392 (1989).

Other researchers have reported that induction of UPase activity increases tumor susceptibility to 5′-deoxy-5-flurouridine. See, e.g., H. Eda et al., “Cytokines induce uridine phosphorylase in mouse colon 26 carcinoma cells and make the cells more susceptible to 5′-deoxy-5-flurouridine,” Jpn. J. Cancer Res., vol. 84, pp. 341-347 (1993); and S. Ikemoto et al., “Augmentation of antitumor activity of 5′-doxy-5-fluorouridine and thymosin fraction 5 in mouse bladder cancer cells in vitro and in vivo,” Cancer Lett., vol. 145, pp. 121-126 (1999).

Increased UPase expression in tumor cells has been associated both with metastasis to lymph nodes and with lower overall survival in SCC patients. See H. Miyashita et al., “Uridine phosphorylase is a potential prognostic factor in patient oral squamous cell carcinoma,” Cancer, vol. 94, pp. 2959-2966 (2002).

The gene expression profiles of four SCC head and neck tumors have been analyzed using a microarray chip with 1187 oligonucleotides. See U.S. patent application Ser. No. 2003/0,175,717; and E. Hanna et al., 2001. From that study, 59 oligonucleotides were identified for use in a molecular chip to predict response of the tumors to radiation by comparing the gene expression profile of the unknown tumor to that of four tumors using cluster analysis. None of the 59 genes in this set was identified as a sole predictor of radiation response. Of the 59 listed genes, 22 were characterized as being radiation-resistant genes, and 37 as radiation-sensitive genes. UPase was characterized as one of the “radiation-resistant” genes.

The only molecular marker currently used as a sole predictor of response to concomitant chemoradiation treatment is EGFR. There exists a need for additional, more accurate molecular markers for the prognosis of treatment with a chemotherapeutic agent or radiation therapy, or concomitant chemoradiation therapy to assist the physician in making recommendations for tumor treatment.

I have discovered that uridine phosphorylase is a surprisingly reliable molecular marker in tumor cells to predict the response of the tumor cells, e.g., squamous cell carcinoma, to treatment with a chemotherapeutic agent and/or radiation therapy. A high level of uridine phosphorylase is a relatively accurate predictor of resistance of the tumor cell to chemoradiation therapy. Surprisingly, this molecular marker by itself was more accurate than other well-known molecular markers for the prediction of the concomitant chemoradiation response. This marker may be used as a prognostic factor on several cancers, including head and neck cancer, skin cancer, ovarian cancer, lung cancer, colon cancer, esophageal cancer, melanoma, and adenocarcinoma. There appear to be no prior suggestions for comparing UPase levels in untreated tumor cells to the sensitivity of the tumor cells to chemoradiation therapy. I have discovered the expression of an additional ten genes that may also be used to predict the response of cancer cells to chemoradiation therapy, i.e., namely, expression of tissue specific extinguisher 1, 60S ribosomal protein L10, C-myc purine-binding transcription factor puf, 60S ribosomal protein L32, metalloproteinase inhibitor 1 precursor, early growth response alpha, early growth response protein 1, bone proteoglycan 1 precursor, BIGH3, and interleukin-6-precursor.

### BRIEF DESCRIPTION OF DRAWINGS

The file of this patent contains at least one drawing executed in color. Copies of this patent with color drawings will be provided by the Patent and Trademark Office upon request and payment of the necessary fee.

FIG. 1 illustrates a heatmap that summarizes the expression levels for eleven genes (listed in Table 1) from RNA isolated from tumor samples from patients with chemoradiation (CR)-resistant squamous cell carcinoma (SCC) and patients with CR-sensitive SCC, wherein a green color represents underexpression and a red color represents overexpression as compared to the average expression level in each gene for all patients assayed.

FIG. 2 illustrates the results of a Northern blot analysis comparing gene expression levels of seven genes as determined from RNA isolated from tumors from six patients with CR-resistant SCC and six patients with CR-sensitive SCC.

### EXAMPLE 1

### Gene Expression Distinguishing ChemoRadiation-Resistant and ChemoRadiation-Sensitive SCC Tumors

The level of expression of several genes was determined from RNA samples collected from eighteen patients prior to any treatment—9 patients with tumors determined to be chemoradiation-resistant and 9 patients with tumors determined to be chemoradiation-sensitive.

RNA Isolation.

Squamous cell carcinoma (SCC) biopsy tissues were obtained from eighteen patients at the University of Arkansas for Medical Sciences, with a protocol approved by the local Institutional Review Board. Snap-frozen tumor tissues were homogenized in TRIzol reagent (Life Technologies, Inc.; Rockville, Md.) with a bead-beater to extract total RNA as described in J. Elek et al., “Microarray based expression profiling in prostate tumors,” In Vivo, vol. 14, pp. 173-182 (2000). All patients were then treated for 6 weeks with a daily dose of 1.8 to 2.0 Gy (for 5 days of the week) for a total radiation dose of about 68-72 Gy. Concurrently, the patients were treated with a total dose of both cisplatin (25 mg/m² body surface area) and 5-fluorouracil (1000 mg/m² body surface area) in three to four cycles of chemotherapy. A “chemoradiation-sensitive tumor” was defined as one in which no tumor was evident at the end of the 6 weeks. A “chemoradiation-resistant tumor” was defined as one in which the tumor size did not decrease by more than about 40% at the end of 6 weeks.

Atlas™ cDNA Array Analysis.

Extracted RNA (30 μg) was treated with 10 units of DNase I (MessageClean Kit, GenHunter Corp., Nashville, Tenn.) for 30 min at 37° C. to digest any contaminating DNA. An aliquot of the resultant RNA (“total RNA”; 5 μg) from each sample was converted into ³²P-labeled first-strand cDNA by reverse transcription using gene-specific primers, according to the manufacturer's specifications (Clontech Laboratories, Inc., Palo Alto, Calif.). Probes were purified and hybridized to the filter array overnight at 68° C. Filters were washed and exposed to Storage Phosphor Screen (Molecular Dynamics, Sunnyvale, Calif.), and differences in signals among the samples were scanned by a PhosphorImager analyzer (Model 445 SI, Molecular Dynamics, Sunnyvale, Calif.) and analyzed using Atlas Image™ 1.5 software (BD Biosciences Clontech, Palo Alto, Calif.). The samples were normalized by exposing filters hybridized with sensitive and resistant samples in a manner such that both filters produced a baseline signal intensity, as determined by eight house-keeping genes. The filters, containing 1,176 unique human cancer-related oligonucleotides, are available from BD Biosciences Clontech.

Statistical Analysis and Generation of Heat Map.

A Student's T-test was performed for each analyzed gene for each of the 18 tumor samples removed prior to chemoradiation treatment; half of the samples were from patients with chemoradiation-resistant tumors and half from patients with chemoradiation-sensitive tumors samples. Genes were identified whose expression levels differed between these two groups at a P value≦0.1 using the Student's T-test. Only 11 genes demonstrated a significant difference at this level. These 11 genes are listed in Table 1.

TABLE 1
Selected molecular markers for prediction of chemoradiation response of
SCCHN patients

| | | Average | Average | | |
|---|---|---|---|---|---|
| Gene | | Expression | Expression | Expression | P |
| Code | Genes | In CR/SP | In CR/RP | ratio R/S | value |
| B08c | Tissue-specific extinguisher 1 (TSE1) | 132 | 180 | 1.36 | .034 |
| F01k | 60S ribosomal protein L10 | 933 | 1506 | 1.61 | .061 |
| A09b | C-myc purine-binding transcription factor | 1015 | 1385 | 1.36 | .069 |
| | puf | | | | |
| F05k | 60S ribosomal protein L32 | 742 | 1271 | 1.71 | .101 |
| E10j | Metalloproteinase inhibitor 1 precursor | 259 | 612 | 2.36 | .073 |
| F06n | Early growth response alpha | 101 | 156 | 1.54 | .070 |
| C12j | Early growth response protein 1 | 188 | 270 | 1.44 | .107 |
| E04n | Bone proteoglycan 1 precursor | 71 | 153 | 2.15 | .071 |
| F14e | BIGH3 | 186 | 429 | 2.31 | .090 |
| E10f | Interleukin-6 precursor | 60 | 121 | 2.02 | .103 |
| F09c | Uridine phosphorylase (UPase) | 47 | 134 | 2.85 | .027 |

CR/SP, chemoradiation/sensitive patients
CR/RP, chemoradiation/resistant patients
R/S, average expression intensity of resistant patients divided by the expression intensity of sensitive patient
P value in the right column was calculated by unpaired t-test
F test, P < 0.001 for the expression intensities of all 11 genes

Expression levels in only two of the 11 genes, UPase and TSE1, demonstrated a significant difference at the P<0.05 level. (Table 1). Of these two genes, only UPase expression differed between the groups by a factor greater than 2. UPase was further analyzed by Northern Blot, as described below in Example 2. When the expression levels for all 11 genes were considered together using an F test, there was a significant difference between the chemoradiation-resistant and chemoradiation-sensitive patients (P<0.001).

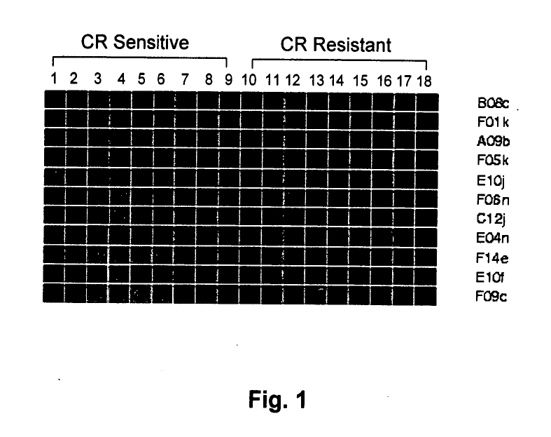

The eleven genes shown in Table 1 were analyzed further. Gene expression values were normalized as described above. These values were analyzed using GeneSifter.Net, microarray analysis software from VizXlabs (Seattle, Wash.). In the software program, the pattern navigation option was selected to generate a heatmap summarizing the expression profiles for each gene for each sample. The heat map generated is shown in FIG. 1, where green color represented downregulation and red color represented upregulation compared with the average expression for each gene for all eighteen patients assayed. Patient patterns were reordered by Euclidean distance to be easily visualized. In FIG. 1, the columns represent the 11 gene expression profiles for each patient, and the rows represent the gene expression variation among the 18 patients.

As shown in FIG. 1, these 11 selected genes could be used collectively to predict a response to treatment with a chemotherapeutic agent or radiation therapy. Oligonucleotides corresponding to these 11 genes will be used in a mini-array chip for predicting the chemoradiation response of SCC or other cancer patients. Such chips may be manufactured through means otherwise known in the art. See, e.g., G. M. Grant et al., “Microarrays in cancer research,” Anticancer Res., vol. 24, pp. 441-118 (2004).

### EXAMPLE 2

### Prognosis by Uridine Phosphorylase Expression Alone

To demonstrate the value of UPase as a sole prognostic marker for chemoradiation response, the level of UPase gene expression was compared by Northern Blot with other known molecular markers, including markers for radiation therapy (e.g., ERBB-2, EGFR, BC12, VEGF, cyclin D1, and BCL2) and for chemoradiation therapy (e.g., EGFR). For this experiment, RNA samples from 6 chemoradiation-resistant SCC head and neck tumors and 6 chemoradiation-sensitive SCC head and neck tumor were used. These twelve samples were a subset of the samples used in Example 1, chosen because sufficient sample remained for this assay. RNA was collected as described in Example 1.

Northern Blot Analysis.

A uridine phosphorylase gene clone was purchased from Open Biosystems (Huntsville, Ala.). The template for probing uridine phosphorylase RNA was obtained by polymerase chain reaction (PCR) using primers SVM31 (5′ ggaatggcggccacggg 3′; SEQ ID NO: 1) and SVM32 (5′ caaggcccagctcttgcacca 3′; SEQ ID NO: 2). The template for probing VEGF (vascular endothelial growth factor) RNA was amplified from pVEGF (BD Pharmagen-Clontech) using primers SVM38 (5′ gcagctactgccatccaatc 3′; SEQ ID NO: 3) and SVM39 (5′ ctgcatggtgatgttggact 3′; SEQ ID NO: 4). The template for probing EGFR (epidermal growth factor receptor) RNA was amplified from pEGFR/cDNA3 using primers UAMS165 (5′ ctgctcgagcagcgatgcgaccctcc 3′ SEQ ID NO: 5; ) and SVM7 (5′ acataaccagccacctcctg 3′; SEQ ID NO: 6). The template for probing ERBB-2 (or HER-2/neu; a member of the tyrosine kinase family) RNA was amplified from perbB2/cDNA using primers SVM8 (5′ ggagccgcagtgagca 3′; SEQ ID NO: 7) and SVM9 (5′ gacctgcctcacttggttgt 3′; SEQ ID NO: 8). The template for probing cyclin D1 RNA was amplified from human genomic DNA using primers SVM36 (5′ ccatggaacaccagctcct 3′; SEQ ID NO. 9) and SVM37 (5′ ggacctccttctgcacacat 3′; SEQ ID NO. 10). The template for probing BCL-2 RNA was amplified from human genomic DNA using primers UAMS14 (5′ cgttacttttcctctggg 3′; SEQ ID NO. 11) and SVM35 (5′ ggctgcgaggagaagatg 3′; SEQ ID NO. 12). The template for probing a-actin RNA was obtained from a Strip-EZ™ PCR kit (Ambion, Inc.; Austin, Tex.).

Northern blot was performed as otherwise described in S. Li et al., “Intramuscular electroporation delivery of IFN-alpha gene therapy for inhibition of tumor growth located at a distant site,” Gene Ther., vol. 8, pp. 400-407 (2001). Approximately 15 μg of total RNA from tumor tissues was electrophoresed on a 1% agarose-formaldehyde gel at 60 V for 3 hr. The RNA was then transferred to a BrightStar-Plus ™ membrane (Ambion, Inc.) and hybridized at 42° C. in ULTRAhyb™ (Ambion, Inc.). Membranes were striped using the Strip_EZ™ kit components and rehybridized with α-actin probe as control for RNA loading. Gel-purified PCR products (2-10 ng) were labeled with Strip_EZ™ PCR kit (Ambion, Inc.). A linear probe was synthesized using antisense primer and α-P³² dATP (3000 Ci/mmol; Amersham Bioscience, Piscataway, N.J.), and PCR-amplified at 94° C. for 30sec, 60° C. for 1 min, and 72° C. for 1 min for a total of 35 cycles. The hybridized membranes were exposed to Cyclone Storage Phosphor Screen and analyzed with Cyclone Storage Phosphor System (Perkin Elmer, Boston, Mass.).

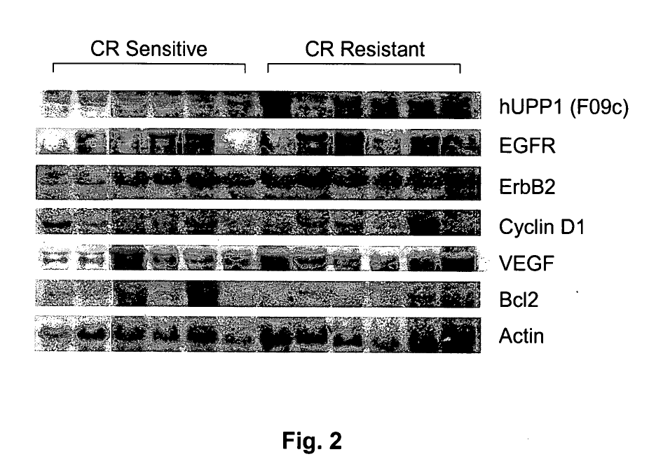

As shown in FIG. 2, a high level of UPase mRNA was detected in 5 of 6 chemoradiation-resistant tumor samples, and a low level of UPase mRNA was detected in all six chemoradiation-sensitive tumor samples. The level of EGFR mRNA was found to be the second-best marker. The expression of the other tested molecular markers had no apparent correlation to the chemoradiation response. (FIG. 2)

Thus UPase may be used as a sole molecular marker to predict the response of SCC to treatment with a chemotherapeutic agent or radiation therapy. In this study, UPase was a better predictor of the chemoradiation response than the other radiation and chemoradiation molecular markers tested.

Other assays for UPase gene expression, e.g., protein concentration or activity could also be used to determine the levels of UPase in tumor cells or tissues. For instance, the amount of UPase could be determined by extracting the protein from homogenized tumor tissue samples using a known protein extraction buffer with protease inhibitors. The UPase concentration could be determined by Western blot or other known techniques for determining concentrations of specific proteins.

Alternatively, the relative amount of UPase could also be determined by an assay for enzyme activity of UPase. The enzymatic activity of UPase may be determined, for example, by the release of uridine diphosphate from uridine triphosphate by techniques known in the art. See, M. Liu et al., “Expression, characterization, and detection of human uridine phosphorylase and identification of variant uridine phosphorylase activity in selected human tumors,” Cancer Res., vol. 58, pp. 5418-5424 (1998).

The complete disclosures of all references cited in this specification are hereby incorporated by reference. In the event of an otherwise irreconcilable conflict, however, the present specification shall control.

I claim:

1. An apparatus comprising a surface, and from 5 to 20 different single-stranded oligonucleotides covalently tethered to said surface; wherein at least one of said nucleotides is adapted to hybridize to an mRNA transcript encoding each of at least 5 different peptides or proteins selected from the group consisting of uridine phosphorylase, tissue specific extinguisher 1, 60S ribosomal protein L10, C-myc purine-binding transcription factor puf, 60S ribosomal protein L32, metalloproteinase inhibitor 1 precursor, early growth response alpha, early growth response protein 1, bone proteoglycan 1 precursor, BIGH3, and interleukin-6-precursor.

2. An apparatus as in claim 1, wherein said nucleotides are adapted to hybridize to an mRNA transcript encoding each of the following peptides or proteins selected from the group consisting of uridine phosphorylase, tissue specific extinguisher 1, 60S ribosomal protein L10, C-myc purine-binding transcription factor puf, 60S ribosomal protein L32, metalloproteinase inhibitor 1 precursor, early growth response alpha, early growth response protein 1, bone proteoglycan 1 precursor, BIGH3, and interleukin-6-precursor.

3. A method for predicting a response of malignant tumor to treatment with a chemotherapeutic agent or radiation therapy, said method comprising taking an RNA sample from the tumor, measuring the expression in the sample of at least one marker gene selected from the group consisting of uridine phosphorylase, tissue specific extinguisher 1, 60S ribosomal protein L10, C-myc purine-binding transcription factor puf, 60S ribosomal protein L32, metalloproteinase inhibitor 1 precursor, early growth response alpha, early growth response protein 1, bone proteoglycan 1 precursor, BIGH3, and interleukin-6-precursor, and comparing said expression pattern with expression patterns of RNA samples from tumors with a known response to treatment with a chemotherapeutic agent or radiation therapy.

4. A method as in claim 3, wherein said treatment is concomitant treatment with a chemotherapeutic agent and radiation therapy.

5. A method for predicting the response of a malignant tumor to treatment with a chemotherapeutic agent or radiation therapy, said method comprising taking an RNA sample from the tumor, measuring the expression of uridine phosphorylase RNA in the sample, and correlating the measured expression of uridine phosphorylase to the likelihood of successful response to treatment with a chemotherapeutic agent or radiation therapy.

6. A method as in claim 5, wherein the tumor is a squamous cell carcinoma.

7. A method as in claim 5, wherein the tumor is selected from the group consisting of head and neck cancer, skin cancer, ovarian cancer, lung cancer, colon cancer, and esophageal cancer.

8. A method as in claim 5, wherein the tumor is head and neck squamous cell carcinoma.

9. A method as in claim 5, wherein said treatment is concomitant treatment with a chemotherapeutic agent and radiation therapy.

10. A method for predicting the response of malignant tumor to treatment with chemotherapeutic agent or radiation therapy, said method comprising taking an RNA sample from the tumor, measuring the expression intensity of tissue-specific extinguisher (TSE1) in the sample, and correlating the measured expression of TSE1 to the likelihood of successful response to treatment with chemotherapeutic agent or radiation therapy.

11. A method as in claim 10, wherein the tumor is a squamous cell carcinoma.

12. A method as in claim 10, wherein the tumor is selected from the group consisting of head and neck cancer, skin cancer, ovarian cancer, lung cancer, colon cancer, and esophageal cancer.

13. A method as in claim 10, wherein the tumor is head and neck squamous cell carcinoma.

14. A method as in claim 10, wherein said treatment is concomitant treatment with a chemotherapeutic agent and radiation therapy.

15. A method for predicting the response of a malignant tumor to treatment with chemotherapeutic agent or radiation therapy, said method comprising taking a tissue sample from the tumor, measuring the expression of uridine phosphorylase in the sample, and correlating the measured expression of uridine phosphorylase to the likelihood of successful response to treatment with chemotherapeutic agent or radiation therapy.

16. A method as in claim 15, wherein the tumor is a squamous cell carcinoma.

17. A method as in claim 15, wherein the tumor is selected from the group consisting of head and neck cancer, skin cancer, ovarian cancer, lung cancer, colon cancer, and esophageal cancer.

18. A method as in claim 15, wherein the tumor is head and neck squamous cell carcinoma.

19. A method as in claim 15, wherein the tumor is head and neck squamous cell carcinoma.

20. A method as in claim 15, wherein said treatment is concomitant treatment with a chemotherapeutic agent and radiation therapy.
